(19) United States
(12) Patent Application Publication
(10) Pub. No.: US 2003/0018374 A1
(43) Pub. Date: Jan. 23, 2003

(54) Treatment probe using RF energy

(76) Inventor: Lonnie E. Paulos, Salt Lake City, UT (US)

Correspondence Address: KIRTON AND MCCONKIE, 1800 EAGLE GATE TOWER, 60 EAST SOUTH TEMPLE, P O BOX 45120, SALT LAKE CITY, UT, 84145-0120, US

(21) Appl. No.: 09/906,430

(22) Filed: Jul. 16, 2001

(51) Int. Cl. A61B 18/18

(52) U.S. Cl. 607/101

(57) ABSTRACT

An RF probe is disclosed for use in treating living tissue under repair or rehabilitation. The probe carries radio-frequency (RF) energy to a treatment zone through one or more prongs that penetrate and pierce the treatment location. The probe has another end, which attaches to an RF generator during operation. An insulator covers most of the probe, from the RF generator attachment end to the base of the prong end. The prongs are generally parallel and have sharpened ends for penetration. An insulating cap is further placed on the RF generator attachment end and is utilized to secure a lead from the RF generator during treatment.

### BACKGROUND

The present invention relates generally to a medical apparatus for the treatment of ligament or ham string grafts and, more specifically, the present invention is related to a medical probe using radio frequency energy for minimizing swelling and otherwise treating ligament or ham string grafts.

The knee joint is one of the strongest joints in the body because of the powerful ligaments that bind the femur and tibia together. Although the knee is vulnerable to injury as a result of the incongruence and proximity of its articular surfaces, the knee joint provides impressive stability due to the arrangement and interacting strength of its ligaments, muscles and tendons.

To a layman, the operation of the human knee resembles the actions of a hinge joint. In reality, however, the knee joint provides complicated mechanical movements and maneuverability far more complex than a simple hinge mechanism in regards to the rotation and gliding motions that may occur at the joint. In addition, the motions of flexing and extending the knee (and, in certain positions, the slight rotation inward and outward of the knee), require a very detailed structural configuration to facilitate the associated, refined mechanical movements of the knee joint.

Structurally, the knee joint comprises two arcuately shaped discs of protective cartilage called menisci, which partially cover the surfaces of the femur and the tibia. The menisci operate to reduce the friction and impact loading between the femur and the tibia during movement of the knee. The knee is also partly surrounded by a fibrous capsule lined with a synovial membrane which secrets a lubricating fluid. Strong ligaments on each side of the knee joint provide support to the joint and limit the side-to-side motion and joint opening of the knee. Fluid filled sacs called bursas are located above and below the patella (kneecap) and behind the knee providing a means of cushioning the kneecap upon impact and helping with joint lubrication. Moreover, the quadriceps muscles run along the front of the thigh to straighten the knee, while the hamstring muscles run along the back of the thigh to bend the knee.

Two intra-articular ligaments of considerable strength, situated in the middle of the joint, are known as the cruciate ligaments. These ligaments are referred to as “cruciate ligaments” because they cross each other somewhat like the lines of the letter “X”. The anterior and posterior cruciate ligaments receive their names in respect to the positioning of their attachment to the tibia. The primary function of the anterior cruciate ligament (ACL) is to provide a means for limiting hyperextension of the knee and preventing the backward sliding of the femur on the tibia plateau. The ACL also assists in limiting any medial rotation of the knee joint when the foot is solidly on the ground and the leg fixed in position. Conversely, the posterior cruciate ligament (PCL) primarily provides a means for preventing hyperflexion of the knee and preventing the femur from sliding forward on the superior tibial surface when the knee is flexed.

Although the structure of the knee provides one of the strongest joints of the body, the knee is usually one of the most frequently injured joints. Athletes and persons who perform tasks requiring a great deal of body rotation are the most susceptible to serious ligament stressing and tearing at the knee joint. Consequently, the growing number of ligament injuries has given rise to considerable innovative activity within the area of orthopedic medicine in an effort to create surgical procedures and devices for replacing and reconstructing torn or dislocated ligaments.

Typically the surgical procedures for ligament replacement and reconstruction involve tissues being grafted from one part of the body (autograft) to the original attachment sites of a torn or dislocated ligament. Once the ligament graft has been harvested, it is then attached via, for example, interference screws to bone tunnels formed at the natural fixation sites of damaged ligament. For example, the replacement of the ACL may involve transplanting a portion of the patellar tendon to the attachment sites of the original ACL to assist in the reconstruction of the ACL in the knee joint.

The expectations of prior art orthopedic procedures typically relate to reconstructing or replacing natural ligaments so as to enable the recipient to return to his or her full range of activity in as short a period of time as possible. To that end, medical researchers have attempted to duplicate the relative parameters of strength, flexibility, and recovery found in natural ligaments of the body. Unfortunately, many of the prior art methods of reconstructing and replacing damaged ligaments have generally proven inadequate for immediately restoring full strength and stability to the involved joint. Furthermore, there has long been a problem of effectively fastening a ligament to a bone surface for the duration of a ligament's healing process, which process involves the ligament graft growing to an adjoining bone mass to restore mobility to the injured joint of an orthopedic patient.

Many ligament replacement procedures typically comprise extensive incisions and openings in the knee to attach a replacement ligament to bone surfaces at the fixation sites of the natural ligament. The ends of a grafted ligament are sometimes secured to exterior bone surfaces by driving stainless steel staples through or across the ligament and into the adjacent bone mass. The legs of the staples are generally adapted for piercing and penetrating tissue and bone mass, while maintaining a ligament at a specified connection site. Other various types of tissue fastening devices, such as channel clamps, were also designed by those skilled in the art. The channel clamps normally differed from the above-mentioned staple arrangement in that the channel clamp fixation devices comprise a plurality of components that do not require clinching in the conventional manner, as when setting a staple into a bone surface.

After a period of time, significant disadvantages emerged wherein a number of the ligament grafts retained in bone mass by the combination drilling/anchor devices began to rupture and tear at their fixation sites around the area where the ligament was in direct contact with the sharp outer edges of the opening of the channel formed in the bone. For example, as replacement ligaments tolerate the stress and strain associated with normal physical activity, the ligament generally begins to fatigue when wearing against the sharp outer edges of a bone channel opening. This form of fatigue typically causes significant damage to the ligament by tearing or cutting into ligament cross-fibers, thus, weakening the connection of the replacement ligament at its reattachment site. Consequently, after a period of time, cross-fiber fatigue, commonly known as “sun-dial” or “windshield wiper” wear, may further result in dislocating the replacement ligament from its original fixation site.

Because of the significant disadvantages associated with “sun-dial” wear or fatigue on replacement ligaments, improved surgical procedures were developed offering arthroscopic-assisted techniques typically including the formation of passages or tunnels through bone mass, wherein natural or synthetic ligaments may be inserted. After the ligament graft has been inserted into the bone tunnel, a ligament anchoring device is generally used to connect one end of a ligament to the exterior of the bone mass. The anchoring means generally requires that the replacement ligament end or ends be advanced beyond the bone tunnel, with each ligament end being bent and secured onto the exterior surface of the bone. Nevertheless, unfavorable disadvantages of ligament bending was observed by those skilled in the art as typically resulting in a force concentration at the location of the ligament bend generally causing the cross-fibers of the ligament to weaken, potentially subjecting the ligament to the possibility of further tearing or rupturing, as in the case of ligament sundial wear. Additionally, exterior devices can rub and cause pain, requiring removal about 10% of the time.

In response to the problems associated with maintaining a replacement ligament graft at a fixation site, additional devices and techniques were developed offering means whereby a ligament may be retained within a bone tunnel by an endosteal fixation device, such as, for example, an interference screw. The threads of the interference screw are typically bored into the bone tunnel for recessed engagement with the attached bone and one end of the ligament graft, while maintaining the ligament at a fixation site within the bone tunnel. Unfortunately, puncturing, piercing and possible tearing sometimes occurs in cross-fibers of the ligament when the ligament is in direct engagement with the sharp threads of the interference screw. In addition, the interference screw typically requires a ligament replacement graft to be attached to its original bone.

During flexion or extension of the ligament, tension loads tend to act against the fixation site of the ligament generally causing strain on the ligament against its fixation site. Under such strain, the spacing of the threads of the interference screw generally effects a pinching or piercing of the ligament which may cause tearing or dislocation of the replacement ligament under the stress associated with normal physical activities. Consequently, when a grafted ligament suffers cross-fiber damage due to puncturing, piercing or tearing, the healing period for the ligament dramatically increases, thereby in effect, increasing the rehabilitation time for the patient to recover.

One of the preferred methods employed by a number of skilled physicians when repairing torn or dislocated ligaments involves the harvesting of an autograft patella tendon bone block for incorporation into a femoral socket. Although the use of a patella tendon bone block provides a number of advantages, especially when dealing with fixation of the replacement ligament, the harvesting of a patella bone block typically results in extensive morbidity to the knee joint, requiring a considerable amount of time for the knee joint to heal, before a patient can resume any normal physical activity.

With each of the preceding procedures, the patient requires rehabilitation time to recover from the surgery on the joint and tendon. This rehabilitation includes convalescing so that the knee and tendon become stronger and usable. The rehabilitation also includes therapy of heat and cold applications. One type of application has been that of using radio frequency to generate heat within the treated area. Typically, two or more probe points at one end are minimally inserted into the treated area, such as at or near the skin surface for treatment, and an RF (radio frequency) is applied through the points to the treated area to provide healing and rehabilitative effects. The method of applying RF energy to the area has been done using electrodes or other RF carriers that are placed on the surface to be treated. Although the RF treatment devices of the prior art have shown progress and usefulness in treating tendons and affected areas of rehabilitation, improvements are still sought to enhance recovery and rehabilitation.

### SUMMARY OF THE INVENTION

According to the present invention, an RF probe is disclosed for use in treating of ligament tissue under repair or rehabilitation. The probe carries radio-frequency (RF) energy to a treatment zone through one or more prongs that penetrate and pierce the treatment location in a localized area below the skin. The probe has another end, which attaches to an RF generator during operation. An insulator preferably covers most of the probe, from the RF generator attachment end to the base of the prong end. A first prong is utilized within one embodiment, but multiple prongs are also contemplated and useful. If more than one prong is used, the prongs are generally parallel and separated by a distance of about 0.100″-0.150″. The probe tip is preferably made of heat treated 400 series stainless steel and the probe shaft is preferably made of 300 series stainless steel. Both stainless steels serve as good conductors of RF energy and are medically sound. An insulating cap can also be placed on the RF generator attachment end and is utilized to secure a lead from the RF generator during treatment. The probe operates preferably in a monopolar mode in a temperature range of about 60 to 66 Celsius and an energy range of about 30 to 40 watts.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 illustrates the RF probe in accordance with the present invention;

FIG. 2 illustrates the RF probe with an insulating cover on the outer surface in accordance with the present invention;

FIG. 3 depicts the RF probe tip as illustrated in FIGS. 1 and 2 in accordance with the present invention;

FIG. 4 illustrates a trident probe tip in accordance with the present invention;

FIG. 5 depicts the shaft of the RF probe illustrated in FIG. 1;

FIG. 6 illustrates anterior flexor tendon histology specimens at a given treatment stage using the RF probe of FIG. 1 and as compared to a control specimen and a specimen treated using a prior art probe; and

FIG. 7 illustrates anterior flexor tendon histology specimens after treatment with the RF probe of FIG. 1 as compared to a control and prior art probe.

### DETAILED DESCRIPTION OF SPECIFIC EMBODIMENTS

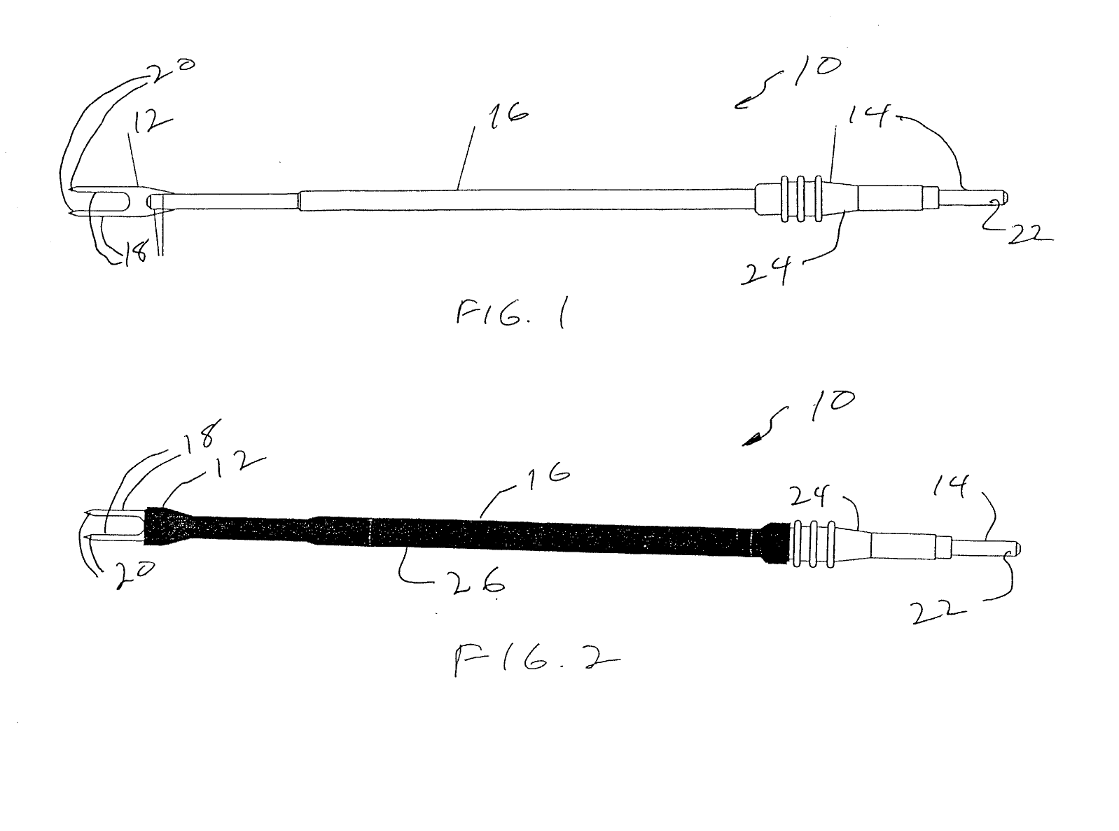

It will be readily understood that the components of the present invention, as generally described and illustrated in the figures herein, could be arranged and designed in a wide variety of different configurations. Thus, the following more detailed description of the embodiments of the system and method of the present invention, and represented in FIGS. 1 through 7, is not intended to limit the scope of the invention, as claimed, but is merely representative of embodiments of the invention.

The specific embodiments of the invention will be best understood by reference to the drawings, wherein like parts are designated by like numerals throughout.

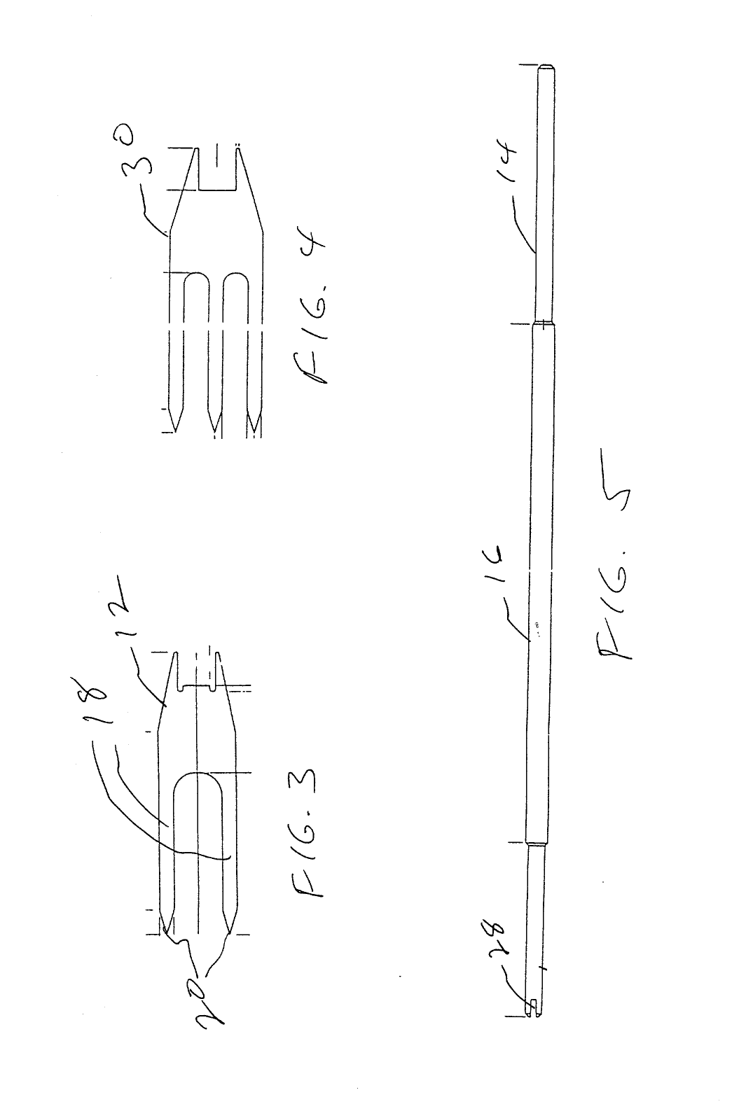

The present invention is directed towards utilizing a novel monopolar RF (radio frequency) probe for use in treating damaged or grafted ligaments, such as those typically performed in anterior cruciate ligament (ACL) or shoulder ligament reconstruction. The RF probe 10 is illustrated in FIGS. 1-5 and includes a first end 12 and a second end 14. A body 16 extends between the two ends. First end 12 is a metallic component, made of 400 series stainless steel and includes a pair of prongs 18, which are generally parallel one to another and are U-shaped or forked and illustrated in greater detail in FIG. 3. First end 12 has a thickness of between 0.050″-0.100″. The end of each prong 18 includes a sharpened point 20, which is utilized to penetrate the location to be treated. Prongs 18 have a separation of approximately 0.120″-0.180″ tip to tip, with a preferred separation of 0.155″. More than two prongs 18 can be provided. For example, a trident prong design 30 is possible as is shown in FIG. 4. One embodiment utilizes a single prong of sufficient width, thickness, and length to effect proper treatment of the treatment area. Each prong 18 is exposed and uninsulated in order to decrease energy density to minimize the likelihood of ablating tissue.

First end 12 attaches to metallic body 16 and second end 14 via reciprocal notches and further secured with laser welds. Body 16 and second end 14 are of unitary construction made from 300 series stainless steel. Other metallic compounds of similar characteristics of that of first end 12 or body 16/second end 14 may be freely substituted. Second end 14 includes an RF connector end 22, which attaches to a RF generator for operation. One example of a RF generator commercially available is one provided by Valley Lab RF. The RF generator is capable of generating power in the range of zero to 60 watts. This results in a temperature for first end 12 ranging from ambient to 100 degrees Celsius. The generator is also capable of changing the temperature gain either in a positive or negative value ranging from 1.0 milliwatt per second to 10.0 milliwatts per second. Other RF generators capable of producing these values are contemplated for use with the RF probe 10 in accordance with principles of the present invention.

Second end 14 further includes an insulation cap or hub 24 (FIGS. 1 and 2), which fits concentric with the axle length of probe 10 and is similar to those typically used with contemporary electrosurgical pencils. The length 16 of probe 10 is covered in an insulating layer insulator 26 to the base of prongs 18 as shown in FIG. 2. In one embodiment, polyolefin shrink tubing is utilized. Other suitable insulating material may be utilized in this application and is left to the skilled artisan for selection. For example, suitable insulating materials capable of withstanding 3000 Volts are desired. Alternatively, a powder coat of an appropriate insulating material can be utilized in place of the polyolefin tubing.

Each probe prong 18 has a length ranging from about 0.250″-0.500″ with 0.400″ being preferred. Further, prongs 18 are generally parallel with one another, but some degree of variance from parallel is allowed as long as the separation between the two prongs falls within the generally accepted dimensions. Also, it is important that the prongs 18 be able to penetrate the treatment location in generally the same direction and parallel one to another. It is only necessary to penetrate the treatment location to a depth of 0.200″-0.500″ when it comes to treating ligaments and hamstrings.

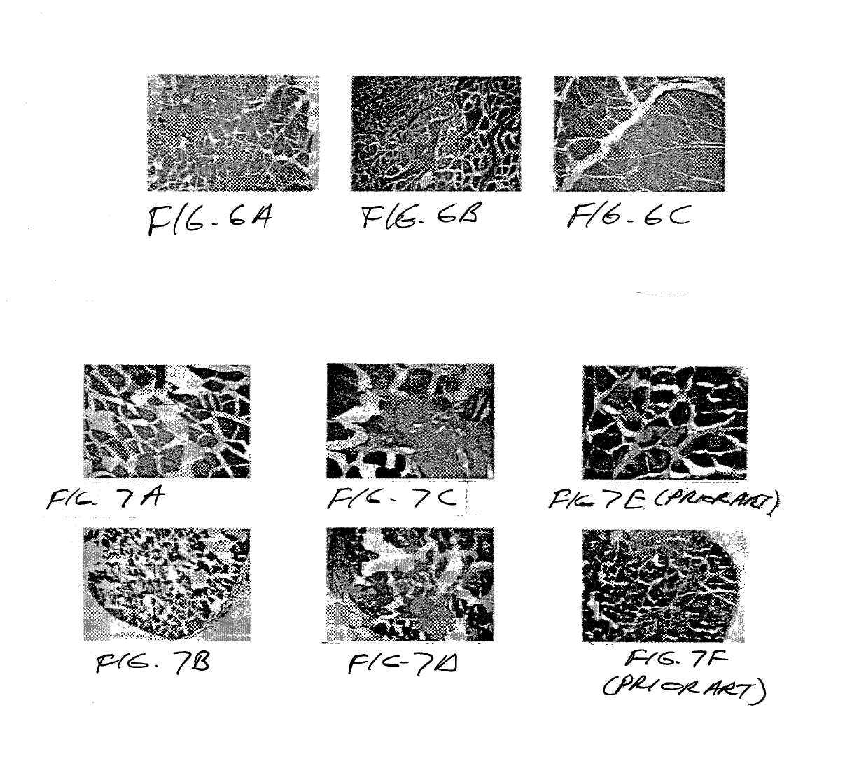

The second end portion 14 of probe 10, which is shown in FIG. 5, has a diameter of about 0.075″-0.100″. The geometric cross-section a profile of probe 10 can be round, oval, rectangular, square, diamond shape, octagonal, or any other geometrical suitable for use. A notch 28 is in first end 12 for receiving first end 12, which has a similar notch 34 (FIG. 3).

Laboratory testing was done with the RF probe 10 in comparison to a prior art probe manufactured by Oratec. The subjects were sheep due to the similarity of the ovine anterior cruciate ligament to the human ACL. RF probe 10, along with the prior art probe, were utilized to attempt to minimize damage to an ACL or hamstring graft after ACL reconstruction and during rehabilitation of the same. Typically, the RF probe provides shrinkage of the damaged ACL. In the experiment, the amount of tendon shrinkage, depth of heat penetration, and histological effects within the tendon were examined. The prior art probe is an Oratec TAC-S probe, which is commercially available and dissimilar to the probe of the present invention. The experimentation and research was accomplished on tissues with properties similar to human ACL's and hamstring tendons to assess the effects of the two probes in applications related to ACL reconstruction. The probe 10 heats the ligament tissue, which typically consists of collagen, and causes it to shrink, thereby tightening what is otherwise a lose ligament. The treatment further consists of providing a rest period, rehabilitation of the affected region, and healing. While this procedure is typically performed on the knee or shoulder, it is equally applicable to any situation where various tissues hold tension between one bone to another.

For the ACL and flexor tendon procedures, the RF generator was used in the cut mode at a gain of a selected value. Utilizing the cut mode will make the probe less likely to generate sparks or arcing. The ACL was stabbed eight times, equally spaced from tibial insertion to femoral insertion. The anterior flexor tendon was stabbed six times, equally spaced along the specimen's pre-treatment length. For all stabs, the surgeon aimed to achieve a depth of penetration near the middle of the tissue. The stab technique simulates “paint-brushing” over the entire capsular surface by affecting as much of the surface as possible via repeated and closely-spaced stabs.

The prior art probe, the Oratec TAC-S probe, had a probe tip temperature set at 65 degrees C. and the RF generator power was set at 30 watts. The positive temperature gain constant was set at 8.0 milliwatt per second, while the negative gain constant was set at 0.8 milliwatt per second. Other testing was performed at a probe tip temperature of 64° C. and the RF generator power set at 40 watts. These were considered to be optimal settings for shrinking the subject anterior flexor tendon.

After a twelve week recovery period, a second RF treatment was performed on the samples. The treatment application was placed in locations different from the original treatment application first performed. Subsequently, the treated tendons were recovered for examination. The control group was merely stabbed with either RF probe without any RF energy being applied thereto. The ACLs that had been stabbed eight times without RF applied appeared mostly normal. In contrast, the ACLs treated with RF were scarred the entire length of the ligament. The tip temperature was set in average of 55° C.-70° C. with 63-66° C. optional. The power applied ranged from 20-60 watts with 30-40 watts preferred. Successive or later treatments would be performed in the same manner using the same temperature and power ranges.

The morphological injury induced by the two methods of treatment, RF power versus stabbed only without power, were not similar. In the RF tissues, there was a point of moderate necrosis on the edge of the ligament as shown in FIG. 6, which specimens are under magnification of 100×. The control sample is shown in FIG. 6A while the stabbed only ligament is shown in FIG. 6B, and the specimen treated with RF probe 10 and applied energy is shown in FIG. 6C. In radial fashion, a zone of less-damaged tissue extended from the area of greatest injury. The injury gradually faded into normal tissue. In the tissue that was stabbed without RF (FIG. 6B), there was very slight necrosis around an apparent puncture point. Most of the reaction was composed of hypercellularity with focal fibrosis. The lesion grades established for the RF treated tissue were compared to the grades for the tissue that were stabbed without RF applied. The RF treated tissues utilizing probe 10 had an average grade of 3.0 while tissues that were stabbed without RF had an average grade of 1.0.

Next, mechanical testing of the ACLs was then performed. The test involved ACLs from the same specimen, but taking either the right or left ACL for actual test. Thus, specimen 1 includes left (L) and right (R) ACLs. The average ultimate loads for the treated ACLs and untreated ACLs are shown in Table 1 below.

TABLE 1

| | CONTROL/ | ULTIMATE | STIFFNESS | FAILURE |
|---|---|---|---|---|
| SPECIMEN | RF | LOAD (N) | (N/mm) | MECH. |
| Specimen 1L | Control | 1197 | 210.0 | Midsubstance |
| Specimen 1R | RF (probe | 609 | 181.3 | Midsubstance |
| | 10) | | | |
| Specimen 2L | Control | 1568 | 330.0 | Avulsion |
| Specimen 2R | RF (probe | 996 | 206.1 | Midsubstance |
| | 10) | | | |
| Specimen 3L | Control | 2093 | 374.2 | Midsubstance |
| Specimen 3R | RF (probe | 1617 | 296.3 | Midsubstance |
| | 10) | | | |

As shown, the untreated ACLs failed at a significantly higher ultimate load than the treated ACLs. The average stiffness for the treated ACLs and untreated ACLs also differed. There was no significant difference in the stiffness for the treated and untreated ACLs. The point of failure of the six ACLs tested was at the midsubstance of the ligament. One untreated ACL failed by avulsion of the ligament from the tibia.

Next, the anterior flexor tendon length and cross-sectional areas were measured to determine what changes had occurred. The average length change for the anterior flexor tendons treated under the probe of the present invention versus that of the prior art (Oratec probe) are given in Table 2 below.

TABLE 2

| | RF | PRIOR | RF | |
|---|---|---|---|---|
| | PROBE 10 | ART | PROBE 10 | PRIOR ART |
| | ΔLENGTH | ΔLENGTH | ΔAREA | ΔAREA |
| SPECIMEN | (mm) | (MM) | (mm²) | (mm²) |
| Specimen (92) | 5.00 | 5.33 | 7.97 | 4.23 |
| Specimen 2 | 6.60 | 10.67 | 8.87 | 2.02 |
| Specimen 1 | 11.43 | 7.62 | 15.58 | 2.77 |
| Specimen 3 | 12.19 | 8.13 | 16.39 | 10.68 |
| Specimen | 7.37 | 8.13 | 5.63 | 3.90 |
| (6557) | | | | |

There was no significant difference in the length change caused by these two methods. The average increase in cross-sectional area was much greater utilizing probe 10 than was achieved utilizing the prior art probe. Thus, the probe 10 caused a significantly larger cross-sectional area change than that of the prior art device.

Next, the anterior flexor tendon histology was secured. The morphological injury induced at the two methods of treatment was similar, as shown in FIG. 7. FIGS. 7A and 7B show the control tendon at 100× and 40× magnification, respectively. FIGS. 7C and 7D show the RF probe 10 treated tendon at 100× and 40× magnification, respectively. FIGS. 7E and 7F show treatment with the prior art (Oratec) probe at 100× and 40×, respectively. At the application point, there was usually marked tissue damage characterized by liquefaction necrosis of collagen bundles and surrounding connective tissue. The issue damage extended in a radial fashion from the application point into the surrounding tissue. Depending on the level of energy applied, the cells adjoining the application point exhibited coagulative necrosis, which progressively decreased in severity in zones distant from the application point. In slightly injured tissues, a rim of normal collagen was present along the edge of the tendon opposite the point of energy application. In the most severely injured tendons, no normal collagen remained. The average lesion grade for the anterior flexor tendons treated with probe 10 were about 3.2±1.3 and for the prior art probe, 2.6±1.1. There was no significant difference in the lesion grade observed for these two treatments (p=0.591)

The experiment demonstrated that the application of RF to an ACL shows healing benefits. The histology results also make it clear that the effects seen are caused by the application of RF, not just stabbing the tissue. Although the ACLs in which RF was applied were 34 percent weaker after a 12 week period, there was no significant difference in their stiffness as compared to a healthy, untreated ACL. Mechanical testing results suggest that the shrunken ACL would act as normal after 12 weeks as long as a maximum load is not placed thereon. It is clear from the histology that after 12 weeks, there is still a noticeable difference in the lesions caused by the RF as compared to those caused by stabbing the ACL. These treated tissues were also much different than the untreated ACLs. From the experimentation, it was determined that it takes longer than 12 weeks for full recovery of the ACL after it has been shrunk using RF.

There was not a significant statistical difference in the length of change for morphological and histological results caused by probe 10 of the present invention versus the prior art probe. However, although the point of application was different for the two procedures, both procedures heated enough surrounding tissue to cause similar results in the sample anterior flexor tendon. The lack of a significant difference in the length change and histological results may be a result of the size of the anterior flexor tendons or the sample size. The higher average length change as well as the higher average lesion rating achieved by probe 10 does show a difference over that of the prior art. This difference is also supported by the significant difference in area increase caused by the use of probe 10 as opposed to the prior art probe. Probe 10 provides a more widespread, thorough burn of the tissue causing increased length shrinkage in cross-sectional area expansion. This results in greater and faster shrinking of most tissues, and especially those tissues having a larger cross-sectional area.

The use of probe 10 may also be applied to shoulder treatment, elbow treatment, or ankle treatment. Specifically, the treatment is useful whenever various tissues that need to hold tension between one bone to another need tightening.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed is:

1. A probe for medically treating living tissue with radio frequency energy, comprising:
a first end capable of attachment to an RF generator;
a second end having a prong of a length sufficient to penetrate at least 6 mm through a treatment area of a patient;
a body, joining the first end to the second end, to carry RF energy from the RF generator to the second end during operation; and
an insulator, covering the body and at least a portion of the first end.

2. The probe of claim 1, wherein the prong has a length of at least 10 mm.

3. The probe of claim 1, wherein the first end and body have a substantially round cross-sectional configuration.

4. The probe of claim 1, wherein said second end operates in a temperature range of about 60° to 66° Celsius.

5. The probe of claim 1, wherein an insulator cap is placed on at least a portion of first end and also attaches to the RF generator.

6. The probe of claim 1, wherein the probe operates in a power range of generally 1.0 to 10.0 milliwatts.

7. The probe of claim 1, wherein the prong has a generally rectangular cross section and pointed tips.

8. The probe of claim 1, wherein the insulator extends to a base of the second end that meets the body.

9. A probe for medically treating living tissue with radio frequency energy, comprising:
a first end capable of attachment to an RF generator;
a second end having at least two prongs separate and substantially parallel one another;
a body, joining the first end to the second end, to carry RF energy from the RF generator to the second end during operation; and
an insulator, covering the body and at least a portion of the first end.

10. The probe of claim 9, wherein the two prongs are separated by a distance of about 0.100″-0.150″.

11. The probe of claim 9, wherein the first end and body have a substantially round cross-sectional configuration.

12. The probe of claim 9, wherein said second end operates in a temperature range of about 60° to 66° Celsius.

13. The probe of claim 9, wherein an insulator cap is placed on at least a portion of first end and also attaches to the RF generator.

14. The probe of claim 9, wherein the probe operates in a power range of generally 1.0 to 10.0 milliwatts.

15. The probe of claim 9, wherein each prong has a generally rectangular cross section and pointed tips.

16. The probe of claim 9, wherein the insulator extends to a base of the second end that meets the body.

17. A method for medically treating living tissue with radio frequency energy, comprising:
inserting a treatment prong device within the area to be treated; and
applying an RF frequency to the treatment area through the prong at a given energy level and for a given length of time.

18. The method of claim 17, wherein the treatment prong device comprises at least two prongs separated by a distance of about 0.100″-0.150″.

19. The method of claim 17, further comprising the steps of:
inserting the treatment prong device at a second location adjacent the insertion treatment area; and
applying the RF frequency to the second location through the prong at the given energy level and for the given length of time.

20. The method of claim 17, wherein the treatment prong device operates in a temperature range of about 60° to 66° Celsius.

21. The method of claim 17, wherein the treatment prong device operates in a power range of generally 1.0 to 10.0 milliwatts.

22. The method of claim 19, wherein each insertion is uniformly spaced apart from one another.

23. The method of claim 19, wherein a plurality of insertions are placed adjacent one another and spaced apart within a given range.

24. The method of claim 17, wherein the depth of insertion ranges from 0.2″-0.5″.

25. The method of claim 17 further comprising the steps of:
resting the treatment area for a given length of time; and
retreating the treatment area with the treatment prong by reinserting it and reapplying the RF energy thereto.
